SEQUENCE LISTING (4 sequences)

SEQ ID NO: 1 - protein, 106 aa, Homo sapien
EKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 2 - DNA, 318 bp, Homo sapien
gagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagacagaagaaactgtggctacccgggcatcagccccgaggaatgcgcctctcggaagtgctgcttctccaacttcatctttgaagtgccatggtgcttcttcccgaagtctgtggaagactgccattac

SEQ ID NO: 3 - DNA, 18 bp, Artificial Sequence
  Synthetic
ctattttcccttcttacg

SEQ ID NO: 4 - DNA, 56 bp, Artificial Sequence
  Synthetic
taatcttagtttctagacttagtaatggcagtctctcacagacttcgggaagaagc

(19) United States
(12) Patent Application Publication
(10) Pub. No.: US 2006/0111278 A1
(43) Pub. Date: May 25, 2006

(54) TFF peptides

(76) Inventors: Lars Thim, Gentofte (DK); Soren E. Bjern, Lyngby (DK); Asser Sloth Andersen, Herlev (DK); Steen Seier Poulsen, Allered (DK)

Correspondence Address: NOVO NORDISK, INC.;PATENT DEPARTMENT, 100 COLLEGE ROAD WEST, PRINCETON, NJ, 08540, US

(21) Appl. No.: 11/002,619

(22) Filed: Dec. 2, 2004

Related U.S. Application Data
(63) Continuation of application No. 10/012,076, filed on Dec. 7, 2001, now abandoned.
(60) Provisional application No. 60/257,506, filed on Dec. 22, 2000.
(60) Provisional application No. 60/303,246, filed on Jul. 5, 2001.

(30) Foreign Application Priority Data
Dec. 8, 2000 (DK) PA 2000 01847
Dec. 9, 2000 (DK) PA 2000 01850
Jun. 14, 2001 (DK) PA 2001 00927

(51) Int. Cl. A61K 38/17

(52) U.S. Cl. 514/8; 514/12

(57) ABSTRACT

A trefoil factor peptide.

### CROSS REFERENCE TO RELATED APPLICATIONS

This application claims priority under 35 U.S.C. 119 of U.S. provisional application Nos. 60/257,506 filed on Dec. 22, 2000, 60/303,246 filed on Jul. 5, 2001 and Ser. No. 10/012,076 filed Dec. 7, 2001; and Danish application nos. PA 2000 01847 filed on Dec. 8, 2000, PA 2000 01850 filed on Dec. 9, 2000 and PA 2001 00927 filed on Jun. 14, 2001, the contents of which are fully incorporated herein by reference.

### FIELD OF THE INVENTION

The present invention relates to novel trefoil factor (TFF) peptides, in particular TFF2 peptides, a method for preparing the TFF peptides, a pharmaceutical composition comprising the TFF peptides, which TFF peptides are for increasing the viscosity of mucus layers in mammals and for the use in the treatment of conditions in mammals with damaged or abnormal mucus layers, e.g. in the gastrointestinal tract, including mouth, oesophagus, stomach, small and large intestine and colon, the respiratory passages, the eye, the urinary system, including the bladder and the cervix uteri.

### BACKGROUND OF THE INVENTION

TFF peptides form a family of peptides found mainly in association with the gastrointestinal tract. Mammalian TFF peptides contain one or more characteristic trefoil domains each of which is made up of a sequence of 38 or 39 amino acid residues in which 6 half-cystine residues are linked in the configuration 1-5, 2-4 and 3-6 thus forming a characteristic trefoil structure.

The mammalian TFF peptides known at present contain either one or two trefoil domains. The mammalian TFF peptides containing one domain are the breast cancer associated pS2 peptide (TFF1) so far known from human, mouse and rat and intestinal trefoil factor, ITF (TFF3) so far known from human, mouse and rat. Spasmolytic polypeptide (TFF2) which contains two trefoil domains has been described from man, pig, rat and mouse. In humans the three TFF peptides hTFF1 (hpS2), hTFF2 (hSP) and hTFF3 (hITF) are all expressed under normal conditions in the gastrointestinal tract: TFF1 and TFF2 in the epithelial mucosal layer of the stomach and TFF3 in the epithelial mucosal layer of the small intestine and colon.

The physiological function of the TFF peptides is not very well understood. Increased expression of TFF peptides in the gastrointestinal tract has been reported in several conditions such as inflammatory bowel disease and ulceration in the stomach and duodenum.

The cloning of rat and human single-domain TFF3 (ITF) and the use of this peptide in the treatment of gastrointestinal injury is described in WO 92/14837.

### DESCRIPTION OF THE INVENTION

The present invention relates to novel TFF2 peptides of the general formula I as shown in FIG. 1, wherein X is as defined below.

The present compounds are useful for repair of damaged or abnormal mucus layers in mammals, such as in the gastrointestinal tract, including mouth, oesophagus, stomach, small and large intestine and colon; the respiratory passages; the eye; and the urinary system, including the bladder and the cervix uteri.

In a broad aspect, the present invention relates to a plurality of TFF2 peptides of the general formula I according to FIG. 1, wherein X represents a covalently attached glycosylation linked to the asparagine on amino acid residue number 15.

In a further aspect, the present invention relates to a plurality of TFF2 peptides of the general formula I according to FIG. 1, wherein X is independently selected from sugar residues and oligosaccharides.

In a further aspect, the present invention relates to a plurality of TFF2 peptides with an amino acid sequence of SEQ ID NO:1 comprising disulphide bonds between Cys6-Cys104, Cys8-Cys35, Cys19-Cys34, Cys29-Cys46, Cys58-Cys84, Cys68-Cys83, and Cys78-Cys95 and wherein a moiety X independently selected from sugar residues and oligosaccharides is covalently attached to Asn15.

In a further aspect, the present invention relates to a plurality of TFF2 peptides with an amino acid sequence of SEQ ID NO:1 comprising disulphide bonds between Cys6-Cys104, Cys8-Cys35, Cys19-Cys34, Cys29-Cys46, Cys58-Cys84, Cys68-Cys83, and Cys78-Cys95 and wherein a moiety X covalently attached to Asn15 is characterized by the glycosylations produced by expression of the TFF2 peptides in a eucaryotic host cell.

The term “a plurality of TFF2 peptides”, as used herein, represent a mixture of TFF2 peptides, where the amino acid sequence of the individual molecules within the mixture is the same according to FIG. 1, but where the covalently asparagine linked glycosylation represented by X, may vary among the individual molecules within the mixture. In a special circumstance within this definition all TFF2 peptides in the mixture have the same glycosylation.

This definition is intended to reflect, that production of the TFF2 peptides in a eucaryotic host cell will not produce a homogenous product, but will produce a heterogenous product, where the glycosylation may vary among the individual TFF2 molecules. However it is possible afterwards to isolate the TFF2 peptides with the individual glycosylation forms.

The term “glycosylation”, as used herein, means the post-translational modification of a peptide, wherein a carbohydrate molecule is covalently attached to the peptide. The glycosylation may take place in a eucaryotic host cell, such as yeast or it may be done by chemical linkage in vitro after production of the peptide in a cell, e.g. the peptide could be produced in a bacteria and glycosylated in vitro afterwards.

The term “a sugar” or “sugar residues”, as used herein, represents carbohydrates of primarily hydrocarbon structure containing polar hydroxyl (—OH) groups. Typical sugars include, but are not limited to, six-carbon (hexose) and five-carbon (pentose) sugars, such as glucose, mannose, galactose, fucose, fructose or N-acetylglucosamine. The term “a sugar” or “a sugar residue” is not restricted to monomers containing only one sugar monomer, but may also represent polymers containing more than one sugar monomer, wherein said sugar monomers within the said polymers, may be the same or different.

The term “oligosaccharide”, as used herein, represents a molecule containing 2 to 100 sugar monomers joined in a linear or a branched structure by glycosidic bonds, wherein said sugar monomers within said oligosaccharide, may be the same or different.

In one embodiment X is a sugar residue.

In another embodiment X is an oligosaccharide.

In a further embodiment X is independently selected from (Hex)ₙ or (GlcNAc)₂—Y or mixtures thereof, wherein n is an integer from 1 to 40 and wherein Y is a sugar residue.

The term “(Hex)ₙ”, as used herein, represent sugars of branched or straight hexose glycosyl residues consisting of n hexose monomers, wherein n is independently selected from 1 to 40 and may be a specific integer or an interval within the limits of 1 to 40. Typical hexose residues include, but are not limited to, mannose, glucose, galactose, fucose, fructose and the like. Nonlimiting examples of (Hex)₂ is Man-Glu or Man-Man sugar residues or mixtures thereof. Nonlimiting examples of (Hex)₁₋₄ is a mixture of Man, Man-Man and Man-Gal-Man-Glu sugar residues or a mixture of Glu, Gal-Man-Gal and Man-Gal-Man-Gal sugar residues.

The terms “Man”, “Glu”, “Gal”, as used herein, represents mannose, glucose and galactose respectively.

The term “Hex”, as used herein, represent a hexose. Typical hexose monomers include, but are not limited to, mannose, glucose, galactose, fucose, fructose and the like.

The term “(GlcNAc)₂” represents two residues of N-acetylglucosamine covalently attached in linear arrangement.

The term “GlcNAc”, as used herein, represent N-acetylglucosamine.

In another embodiment X is (Hex)ₙ, wherein n is an integer from 1 to 40, such as from 5-35, 10-25, 12-20 or 13-17.

In another embodiment X is (GlcNAc)₂—Y, wherein Y is an independently selected sugar residue.

In another embodiment X is (GlcNAc)₂-(Hex)ₙ, wherein n is an integer from 1 to 40, such as from 3-34, 5-28, 7-20 or 10-15.

In another embodiment X is (GlcNAc)₂-(Hex)ₙ( (GlcNAc)(Hex) )ₘ, wherein n and m are integers independently selected from from 1 to 40, such as from 1-5, 1-10, 2-30, 3-20, 4-15 or 5-10.

In still another embodiment X is (GlcNAc)₂-(Hex)ₙ( (GlcNAc)(Gal) )ₘ, wherein n and m are integers independently selected from from 1 to 40, such as from 1-5, 1-10, 2-30, 3-20, 4-5or 5-10.

The term “(Hex)ₙ( (GlcNAc)(Hex) )ₘ”, as used herein, represent branched or straight sugar residues consisting of n hexose monomers covalently attached to m (GlcNAc)(Hex) residues, wherein n and m are independently selected from 1 to 40 and may be a specific integer or an interval within the limits of 1 to 40. Typical Hex residues include, but are not limited to, mannose, glucose, galactose, fucose, fructose and the like.

The term “(GlcNAc)(Hex)” as used herein, represent one molecule of a GlcNAc co-valently attached to one molecule of a Hex.

In another embodiment X is (GlcNAc)₂-(Hex)ₙ( (GlcNAc)(Hex)(NeuAc) )ₘ, wherein n and m are integers independently selected from 1 to 40, such as from 1-5, 1-10, 2-30, 3-20, 4-15 or 5-10.

In another embodiment X is (GlcNAc)₂-(Hex)ₙ( (GlcNAc)(Gal)(NeuAc) )ₘ, wherein n and m is integers independently selected from 1 to 40, such as from 1-5, 1-10, 2-30, 3-20, 4-15 or 5-10.

The term “(Hex)ₙ( (GlcNAc)(Hex)(NeuAc) )ₘ”, as used herein, represent branched or straight sugar residues consisting of n hexose monomers covalently attached to m (GlcNAc)(Hex)(NeuAc) residues, wherein n and m are independently selected from 1 to 40 and may be a specific integer or an interval within the limits of 1 to 40. Typical Hex residues include, but are not limited to, mannose, glucose, galactose, fucose, fructose and the like.

The term “(GlcNAc)(Hex)(NeuAc)” as used herein, represent one molecule of a GlcNAc covalently attached to one molecule of a Hex and to a molecule of NeuAc in a linear arrangement.

The term “NeuAc”, as used herein, represent N-acetylneuraminic acid

In a preferred embodiment of the present invention Hex is a mannose.

The term “carbohydrate”, as used herein, represents molecules of hydrocarbon structure containing polar hydroxyl (—OH) groups.

In still another embodiment X is characterized by the glycosylations produced by expression of the TFF2 peptides in a eucaryotic host cell.

In a preferred embodiment X is characterized by the glycosylations produced by expression of the TFF2 peptides in yeast, such as in Saccharomyces cerevisiae.

In another embodiment X is characterized by the glycosylations produced by expression of the TFF2 peptides in a mammalian cell line, such as in a human cell line

In another embodiment X is characterized by the glycosylations produced by expression of the TFF2 peptides in an insect cell line.

In another embodiment X is characterized by the high mannose-type glycosylation as presented in FIG. 7.

In another embodiment X is characterized by the complex-type glycosylation as presented in FIG. 7.

In another embodiment X is characterized by the hybrid-type glycosylation as presented in FIG. 7.

In still another embodiment X is characterized by a mixture of the high mannose-type, and/or the complex-type and/or the hybrid-type glycosylations as presented in FIG. 7.

In another aspect, the present invention relates to a pharmaceutical composition comprising a plurality of TFF2 peptides according to FIG. 1 together with a pharmaceutically acceptable carrier or diluent.

In a further aspect, the present invention relates to a pharmaceutical composition comprising a plurality of TFF2 peptides with an amino acid sequence of SEQ ID NO:1 comprising disulphide bonds between Cys6-Cys104, Cys8-Cys35, Cys19-Cys34, Cys29-Cys46, Cys58-Cys84, Cys68-Cys83, and Cys78-Cys95 and wherein a moiety X independently selected from sugar residues and oligosaccharides is covalently attached to Asn15.

In one embodiment, the present invention relates to a pharmaceutical composition for the treatment of damaged or abnormal mucus layers in mammals, the composition comprising a plurality of TFF2 peptides according to FIG. 1 or a pharmaceutically acceptable salt thereof together with a pharmaceutically acceptable carrier or diluent.

The term “treatment”, as used herein, means the administration of an effective amount of a therapeutically active compound of the invention with the purpose of preventing any symptoms or disease state to develop or with the purpose of curing or easing such symptoms or disease states already developed. The term “treatment” is thus meant to include prophylactic and protective treatment. The symptoms or disease state includes but is not limited to diseases, e.g. gastric ulcers or asthma, inherited biological disorders or conditions induced by damaging by external stimuli, e.g. Inhalation of toxic or acidic chemical.

In another embodiment, the present invention relates to a pharmaceutical composition for the treatment of damaged or abnormal mucus layers in the gastrointestinal tract of a mammal, preferably in a human.

The term “gastrointestinal tract”, as used herein, includes but is not limited to mouth, oesophagus, stomach, small and large intestine and colon.

In a further embodiment, the present invention relates to a pharmaceutical composition for the treatment of damaged or abnormal mucus layers in the respiratory passages of a mammal, preferably in a human.

In a further embodiment, the present invention relates to a pharmaceutical composition for the treatment of damaged or abnormal mucus layers in the eye of a mammal, preferably in a human.

In a further embodiment, the present invention relates to a pharmaceutical composition for the treatment of damaged or abnormal mucus layers in the urinary system of a mammal, preferably in a human.

The term “urinary system”, as used herein, includes but is not limited to the urethra, bladder, ureter, kidneys and the cervix uteri

In a further embodiment, the present invention relates to a pharmaceutical composition for the treatment of damaged or abnormal mucus layers and for oral, nasal, transdermal, pulmonal, or parenteral administration.

In another aspect, the present invention relates to a method for preparing a plurality of TFF2 peptides according FIG. 1, the method comprising culturing a suitable host cell transformed with a DNA sequence encoding a TFF2 peptide under conditions permitting glycosylation, and recovering the resulting glycosylated TFF2 peptides from the culture.

In a further aspect, the present invention relates to a method for preparing a plurality of TFF2 peptides with an amino acid sequence of SEQ ID NO:1 comprising disulphide bonds between Cys6-Cys104, Cys8-Cys35, Cys19-Cys34, Cys29-Cys46, Cys58-Cys84, Cys68-Cys83, and Cys78-Cys95 and wherein a moiety X independently selected from sugar residues and oligosaccharides is covalently attached to Asn15, the method comprising culturing a eucaryotic host cell transformed with a DNA sequence encoding a TFF2 peptide under conditions permitting glycosylation, and recovering the resulting TFF2 peptides from the culture.

In still another aspect, the present invention relates to a DNA construct containing a nucleotide sequence encoding human TFF2 peptide having the amino acid sequence as shown in FIGS. 1 or 3.

In a further aspect, the present invention relates to a DNA construct containing a nucleotide sequence encoding human TFF2 peptide having the amino acid sequence of SEQ ID NO:1.

In one embodiment the DNA construct contains a nucleotide sequence encoding human TFF2 peptide having the amino acid sequence as shown in FIG. 3 from amino acid 1-106.

In a further embodiment the DNA construct containing a nucleotide sequence encoding human TFF2 peptide having the amino acid sequence as shown in FIG. 3 also encodes a leader peptide and a Lys-Arg cleavage site.

In another embodiment the DNA construct containing a nucleotide sequence encoding human TFF2 peptide having the amino acid sequence as shown in FIG. 3 comprises the cDNA sequence from bp 236 to bp 553.

In a further embodiment the DNA construct containing a nucleotide sequence encoding human TFF2 peptide having the amino acid sequence of SEQ ID NO:1 comprises the cDNA sequence of SEQ ID NO:2.

In a preferred embodiment the DNA construct containing a nucleotide sequence encoding human TFF2 peptide having the amino acid sequence as shown in FIG. 3 comprises the complete cDNA sequence from bp 1 to 563.

In still another aspect, the present invention relates to a recombinant vector capable of transforming a host cell, wherein said vector contains a nucleotide sequence encoding human TFF2 peptide having the amino acid sequence as shown in FIG. 3, a promoter for host cell propagation and a selection marker for a cell containing the vector.

In a further aspect, the present invention relates to a recombinant vector capable of transforming in a host cell, wherein said vector contains a nucleotide sequence encoding human TFF2 peptide having the amino acid sequence of SEQ ID NO:1; a promoter for host cell propagation and a selection marker.

The term “vector”, as used herein, means any nucleic acid entity capable of the amplification in a host cell. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The choice of vector will often depend on the host cell into which it is to be introduced. Vectors include, but are not limited to plasmid vectors, phage vectors or cosmid vectors.

In one embodiment of this invention the recombinant vector is capable of transforming a host cell, wherein said vector contains a nucleotide sequence encoding human TFF2 peptide having the amino acid sequence as shown in FIG. 3, wherein the host cell is yeast, preferably Saccharomyces cerevisiae.

In another embodiment of the present invention the recombinant vector is capable of transforming a host cell, wherein said vector contains a nucleotide sequence encoding human TFF2 peptide having the amino acid sequence as shown in FIG. 3, wherein the host cell is a bacteria.

In another embodiment of the present invention the recombinant vector is capable of transforming a host cell, wherein said vector contains a nucleotide sequence encoding human TFF2 peptide having the amino acid sequence as shown in FIG. 3, wherein the host cell is an insect cell.

In still another embodiment of the present invention the recombinant vector is capable of transforming a host cell, wherein said vector contains a nucleotide sequence encoding human TFF2 peptide having the amino acid sequence as shown in FIG. 3, wherein the host cell is a mammalian cell.

In still another embodiment of the present invention the recombinant vector is capable of transforming a host cell, wherein said vector contains a nucleotide sequence encoding human TFF2 peptide having the amino acid sequence as shown in FIG. 3 wherein the host cell is a human cell.

In still another aspect, the present invention relates to a yeast cell transfected with a recombinant vector capable of transforming the yeast cell, wherein said vector contains a nucleotide sequence encoding human TFF2 peptide having the amino acid sequence as shown in FIG. 3, a promoter for yeast cell propagation and a selection marker for a yeast cell containing the vector.

In a preferred embodiment of the present invention, the recombinant vector is a DNA plasmid.

Accordingly, the present invention provides the use of a plurality of TFF2 peptides of the general formula I, as represented in FIG. 1 for the preparation of a pharmaceutical composition for the treatment of damaged or abnormal mucus layers, such as in the gastrointestinal tract, including mouth, oesophagus, stomach, small and large intestine and colon, the respiratory passages, the eye, the urinary system, including the bladder and the cervix uteri.

The TFF2 peptides of the present invention may have one or more asymmetric centres and it is intended that stereoisomers (optical isomers), as separated, pure or partially purified stereoisomers or racemic mixtures thereof are included in the scope of the invention.

Apart from the pharmaceutical use of the compounds of formula I as represented by FIG. 1, they may be useful in vitro tools for investigating conditions in mammals with damaged or abnormal mucus layers.

TFF2 peptides of formula I (FIG. 1) may also be useful in vivo tools for evaluating conditions in mammals with damaged or abnormal mucus layers, such as in the gastrointestinal tract, including mouth, oesophagus, stomach, small and large intestine and colon, the respiratory passages, the eye, the urinary system, including the bladder and the cervix uteri.

The plurality of TFF2 peptides of the present invention have been shown to be useful for the treatment of damaged or abnormal mucus layers in mammals associated with the following diseases: Gastrointestinal disorders such as gastro oesophageal reflux, ulceration, inflammatory bowel disease including Crohn's disease, Sjögren's syndrome, carcinomas such as gastric, pancreatic, ampullary, bronchial or squamous cell carcinomas, Barrett's metaplasia, hiatus hernier or injury to the intestinal tract caused by radiation therapy, bacterial or other infections, etc., airway diseases such as asthma, chronic and acute bronchitis or cystic fibrosis, eye diseases and disorders in the urinary system and the cervix uteri. The advantage over known therapies is treatment with TFF2 peptides represent a specific treatment at the site of injury without major side effects.

In a preferred embodiment, the plurality of TFF2 peptides of the invention have approximate molecular weights between 14000 and 15000.

The present invention also relates to the use of glycosylated K99-TFF2 peptides for improving rheological properties of mucin solutions. Glycosylated K99-TFF2 peptides have by the present inventors been found to increase the viscosity and elasticity of different mucins solutions, which are correlated to physiological and pathophysiological conditions.

The present invention discloses the mechanism by which glycosylated K99-TFF2 peptides exerts their biological activity, which are documented by a direct effect of glycosylated K99-TFF2 peptides on the viscosity and elasticity of mucin solutions. The glycosylated K99-TFF2 peptides significantly increases the viscosity of mucin solutions. The net effect is an increase in the viscosity of several times and can be visualised by the fact that the liquid mucin solution is converted into a more viscous gel-like substance.

When expressed in yeast K99-TFF2 peptides are secreted in a glycosylated and a non-glycosylated form. The glycosylated form generates more viscous gel-like structure as compared to the non-glycosylated.

The glycosylated K99-TFF2 peptides have by the present inventors been found to be usefull for increasing the viscosity and elasticity of mucus layers, which can be used in the treatment of many different indications, where abnormalities in existing mucus layers are present. The advantage over known therapies is that treatment with glycosylated K99-TFF2 peptides represent a specific treatment at the site of injury without major side effects.

For local and luminal applications glycosylated K99-TFF2 peptides can increase the viscosity and elastic properties of mucin in mucus layers, which may be usefull in many different indications:

1) For the treatment of the oral mucosa. Glycosylated K99-TFF2 peptides may be given alone or together with mucus-like preparations to patients with reduced secretion of saliva caused by irradiation therapy, treatment with anticholinergics or in patients with Sjögrens syndrome.

2) For increasing the viscosity of nasal secretions in rhinorrhoea in common cold or allergic rhinitis. Protection of the mucosa of respiratory tract following accidental inhalation of irritants, gases, dusts or fumes.

3) For protection of the distal part of the oesophagus against acid secretions from the stomach in reflux oesophagi's, hiatus hernia, Barrets oesophagus.

4) For the protection of the stomach against acute stress induced gastric ulcers secondary to trauma, shock, large operations, renal or lever diseases, or gastritis caused by treatment with aspirin or other NSAIDS, steroids or by alcohol.

5) For the treatment of acute or prolonged diarrhoea by increasing the viscosity of the intestinal secretions.

6) For the protection of the small intestinal and colonic mucosa in Crohns disease and ulcerative colitis.

7) In eye droplets to increase the viscosity of lacrimal fluid in patients with keratoconjunctivitis sicca/Sjögren's syndrome or “dry eyes” for other reasons.

8) Local application especially in the knee joints to increase the viscosity of the synovial fluid in osteoarthritis and following joint replacement.

Glycosylated K99-TFF2 peptides may also be used for parenteral applications:

Parenteral glycosylated K99-TFF2 peptides are taken up by cells associated with stem cells in the gastrointestinal tract. It can be used for protection of the stomach against stress-induced damage and the stomach and intestine against damage following irradiation or chemotherapy or in the treatment of acute excerbations in ulcerative colitis or Crohn's disease. Injected glycosylated K99-TFF2 peptides are excreted intact in urine and may increase the defence mechanism of the urinary bladder by binding to the layer of mucopolysaccharids that coat the urothelium and thereby interfere with the adherence of bacteria in chronic bladder infections, in patients with catheter or interstitial cystitis, or interfere with the binding of urinary growth factors in papillomas or cancer of the bladder.

Thus, in a further aspect, the present invention relates to a pharmaceutical composition for increasing the viscosity of mucus layers in mammals, the composition comprising a plurality of TFF2 peptides or a pharmaceutically acceptable salt thereof.

In a further aspect, the present invention relates to a pharmaceutical composition for increasing the viscosity of mucus layers in mammals, the composition comprising glycosylated K99-TFF2 peptides or a pharmaceutically acceptable salt thereof.

In a further aspect, the present invention relates to the use of a plurality of TFF2 peptides for the preparation of a medicament.

In a further aspect, the present invention relates to the use of glycosylated K99-TFF2 peptides for the preparation of a medicament.

In a further aspect, the present invention relates to the use of a plurality of TFF2 peptides for the preparation of a medicament for increasing the viscosity of mucus layers in mammals.

In a further aspect, the present invention relates to the use of glycosylated K99-TFF2 peptides for the preparation of a medicament for increasing the viscosity of mucus layers in mammals.

In a further aspect, the present invention relates to a method for in vivo increase in viscosity of mucus layers in a subject, the method comprising administering to the subject a composition comprising
(a) a pharmaceutically acceptable carrier or diluent,
(b) a therapeutically effective amount of a plurality of TFF2 peptides, and optionally
(c) a mucin glycoprotein preparation,

In a further aspect, the present invention relates to a method for in vivo increase in viscosity of mucus layers in a subject, the method comprising administering to the subject a composition comprising
(a) a pharmaceutically acceptable carrier or diluent,
(b) a therapeutically effective amount of glycosylated K99-TFF2 peptides, and optionally
(c) a mucin glycoprotein preparation,

In another aspect, the present invention relates to the use of a plurality of TFF2 peptides for the treatment of conditions with increased viscosity of mucus layers in mammals.

In one embodiment of the invention, the mammal is human.

In another aspect, the present invention relates to the use of glycosylated K99-TFF2 peptides for the treatment of conditions with increased viscosity of mucus layers in mammals.

In one embodiment of the invention, the mammal is human.

In another embodiment the present invention relates to a pharmaceutical composition for local application.

In a further embodiment the present invention relates to a pharmaceutical composition for luminal application.

In a further embodiment the present invention relates to a pharmaceutical composition for parenteral administration.

In a further embodiment the present invention relates to a pharmaceutical composition for oral administration.

In a further embodiment the present invention relates to a pharmaceutical composition further comprising a mucin glycoprotein preparation.

In a further embodiment the present invention relates to a pharmaceutical composition for the treatment of oral mucosa.

In a further embodiment the present invention relates to a pharmaceutical composition for the treatment of patients with reduced secretion of saliva. In one embodiment, the reduced secretion of saliva is caused by irradiation therapy, treatment with anticholinergics or Sjögrens syndrome.

In a further embodiment, the present invention relates to a pharmaceutical composition for the treatment of patients receiving irradiation therapy.

In a further embodiment, the present invention relates to a pharmaceutical composition for the treatment of patients treated with anticholinergics.

In a further embodiment, the present invention relates to a pharmaceutical composition for the treatment of patients with Sjögrens syndrome.

In a further embodiment the present invention relates to a pharmaceutical composition for the treatment of the respiratory passages.

In a further embodiment the present invention relates to a pharmaceutical composition for increasing the viscosity of nasal secretions in rhinorrhoea in common cold or allergic rhinitis.

In a further embodiment, the present invention relates to a pharmaceutical composition for the treatment of patients with common cold.

In a further embodiment, the present invention relates to a pharmaceutical composition for the treatment of patients with allergic rhinitis.

In a further embodiment the present invention relates to a pharmaceutical composition for the treatment of the respiratory tract.

In a further embodiment the present invention relates to a pharmaceutical composition for the treatment of the respiratory tract following accidental inhalation of irritants.

In a further embodiment the present invention relates to a pharmaceutical composition for the treatment of the respiratory tract following accidental inhalation of gases, dusts or fumes.

In a further embodiment the present invention relates to a pharmaceutical composition for the treatment of oesophagus. In one embodiment the present invention relates to a pharmaceutical composition for the treatment of the distal part of the oesophagus.

In a further embodiment the present invention relates to a pharmaceutical composition for protection against acid secretions from the stomach.

In a further embodiment the present invention relates to a pharmaceutical composition for protection against acid secretions from the stomach in reflux oesophagi's.

In a further embodiment the present invention relates to a pharmaceutical composition for protection against acid secretions from the stomach in hiatus hernia.

In a further embodiment the present invention relates to a pharmaceutical composition for protection against acid secretions from the stomach in Barrets oesophagus.

In a further embodiment the present invention relates to a pharmaceutical composition for the treatment of the stomach.

In a further embodiment the present invention relates to a pharmaceutical composition for treatment of stress induced gastric ulcers. In one embodiment the stress induced gastric ulcers is secondary to trauma. In another embodiment the stress induced gastric ulcers is secondary to shock. In a further embodiment the stress induced gastric ulcers is secondary to large operations. In a further embodiment the stress induced gastric ulcers is secondary to renal diseases. In a further embodiment the stress induced gastric ulcers is secondary to lever diseases. In a further embodiment the stress induced gastric ulcers is secondary to treatment with aspirin, other non-steroidal anti-inflammatory drugs (NSAIDS), steroids or alcohol.

In a further embodiment the present invention relates to a pharmaceutical composition for the treatment of diarrhoea.

In a further embodiment the present invention relates to a pharmaceutical composition for the treatment of the small intestinal mucosa.

In a further embodiment the present invention relates to a pharmaceutical composition for the treatment of the colonic mucosa.

In a further embodiment the present invention relates to a pharmaceutical composition for the treatment of Crohns disease.

In a further embodiment the present invention relates to a pharmaceutical composition for the treatment of ulcerative colitis.

In a further embodiment the present invention relates to a pharmaceutical composition for the treatment of the eye.

In a further embodiment the present invention relates to a pharmaceutical composition for increasing the viscosity of lacrimal fluid.

In a further embodiment the present invention relates to a pharmaceutical composition for increasing the viscosity of lacrimal fluid in patients with keratoconjunctivitis sicca.

In a further embodiment the present invention relates to a pharmaceutical composition for increasing the viscosity of lacrimal fluid in patients with Sjögren's syndrome.

In a further embodiment the present invention relates to a pharmaceutical composition for increasing the viscosity of lacrimal fluid in patients with dry eyes.

The term “dry eyes”, as used herein, means any condition where the eyes feels dry.

In a further embodiment the present invention relates to a pharmaceutical composition in eye droplets.

In a further embodiment the present invention relates to a pharmaceutical composition for the treatment of a joint.

In a further embodiment the present invention relates to a pharmaceutical composition for the treatment of the knee joints.

In a further embodiment the present invention relates to a pharmaceutical composition for increasing the viscosity of the synovial fluid.

In a further embodiment the present invention relates to a pharmaceutical composition for increasing the viscosity of the synovial fluid in osteoarthritis.

In a further embodiment the present invention relates to a pharmaceutical composition for increasing the viscosity of the synovial fluid following joint replacement.

In a further embodiment the present invention relates to a pharmaceutical composition for the treatment of the bladder.

In a further embodiment the present invention relates to a pharmaceutical composition for the treatment of patients with catheter.

In a further embodiment the present invention relates to a pharmaceutical composition for the treatment of infections. In one embodiment the infection is a cronic infection of the bladder.

In a further embodiment the present invention relates to a pharmaceutical composition for the treatment of interstitial cystitis.

In a further embodiment the present invention relates to a pharmaceutical composition for the treatment of papillomas.

In a further embodiment the present invention relates to a pharmaceutical composition for the treatment of cancer.

The invention also relates to a method of preparing the compounds mentioned above. The plurality of TFF2 peptides are preferably produced by recombinant DNA techniques. To this end, a DNA sequence encoding the TFF2 peptide may be isolated by preparing a genomic or cDNA library and screening for DNA sequences coding for all or part of the peptide by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the DNA sequence encoding the peptide is preferably of human origin, i.e. derived from a human genomic DNA or cDNA library.

The DNA sequences encoding the TFF2 peptides may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The DNA sequences encoding the TFF2 peptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the TFF2 peptide is operably linked to additional segments required for transcription of the DNA. In general, the expression vector is derived from plasmid or viral DNA, or may contain elements of both. The term, “operably linked“indicates that the segments are arranged so that they function in concert for their intended purposes, e.g. transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the TFF2 peptide in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequence encoding the TFF2 peptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

To direct a TFF2 peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the TFF2 peptide in the correct reading frame. Secretory signal sequences are commonly positioned 5′ to the DNA sequence encoding the peptide. The secretory signal sequence may be that, normally associated with the peptide or may be from a gene encoding another secreted protein.

For secretion from yeast cells, the secretory signal sequence may encode any signal peptide, which ensures efficient direction of the expressed TFF2 peptide into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp.127-137).

For efficient secretion in yeast, a sequence encoding a leader peptide may also be inserted downstream of the signal sequence and upstream of the DNA sequence encoding the TFF2 peptide. The function of the leader peptide is to allow the expressed peptide to be directed from the endoplasmic reticulum to the Golgi apparatus and further to a secretory vesicle for secretion into the culture medium (i.e. exportation of the TFF2 peptide across the cell wall or at least through the cellular membrane into the periplasmic space of the yeast cell). The leader peptide may be the yeast α-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the TFF2 peptide, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

The host cell into which the DNA sequence encoding the TFF2 peptide is introduced may be any cell, which is capable of producing the posttranslational modified TFF2 peptide and includes yeast, fungi and higher eucaryotic cells.

Examples of suitable mammalian cell lines are the COS (ATCC CRL 1650), BHK (ATCC CRL 1632, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding the TFF2 peptide may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the plurality of TFF2 peptides after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The TFF2 peptides produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

In the pharmaceutical composition of the invention, the plurality of TFF2 peptides may be formulated by any of the established methods of formulating pharmaceutical compositions, e.g. as described in Remington's Pharmaceutical Sciences, 1985. The composition may be in a form suited for systemic injection or infusion and may, as such, be formulated with sterile water or an isotonic saline or glucose solution. The compositions may be sterilized by conventional sterilization techniques, which are well known in the art. The resulting aqueous solutions may be packaged for use or filtered under aseptic conditions and lyophilized, the lyophilized preparation being combined with the sterile aqueous solution prior to administration. The composition may contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

The pharmaceutical composition of the present invention may also be adapted for nasal, transdermal or rectal administration. The pharmaceutically acceptable carrier or diluent employed in the composition may be any conventional solid carrier. Examples of solid carriers are lactose, terra alba, sucrose, talc, gelatin, agar, pectin, acacia, magnesium stearate and stearic acid. Similarly, the carrier or diluent may include any sustained release material known in the art, such as glyceryl monostearate or glyceryl distearate, alone or mixed with a wax. The amount of solid carrier will vary widely but will usually be from about 25 mg to about 1 g.

The concentration of the TFF2 peptides in the composition may vary widely, i.e. from from about 5% to about 100% by weight. A preferred concentration is in the range of 50-100% by weight. A unit dosage of the composition may typically contain from about 1 mg to about 200 mg, preferably from about 25 mg to about 75 mg, in particular about 50 mg, of the peptide.

As indicated above, the glycosylated plurality of TFF2 peptides of the invention are believed to be the active forms of the peptides. As such it is contemplated to be advantageous to use for prevention or treatment of conditions in mammals with damaged or abnormal mucus layers. More specifically, it is contemplated for use in the treatment of gastrointestinal disorders such as gastro oesophageal reflux, ulceration, inflammatory bowel disease including Crohn's disease, Sjögren's syndrome, carcinomas such as gastric, pancreatic, ampullary, bronchial or squamous cell carcinomas, Barrett's metaplasia, hiatus hernier or injury to the intestinal tract caused by radiation therapy, bacterial or other infections, etc., airway diseases such as asthma, chronic and acute bronchitis or cystic fibrosis, eye diseases and disorders in the urinary system and the cervix uteri. The dosage of the polypeptide administered to a patient will vary with the type and severity of the condition to be treated, but is generally in the range of 0.1-1.0 mg/kg body weight.

### BRIEF DESCRIPTION OF THE DRAWINGS

The present invention is described in further detail in the examples with reference to the appended drawings wherein

FIG. 1 The structure of the glycosylated human K99-TFF2, where X represents the glycosylation. Disulphide bonds between Cys6-Cys104, Cys8-Cys35, Cys19-Cys34, Cys29-Cys46, Cys58-Cys84, Cys68-Cys83, Cys78-Cys95 are schematically represented.

FIG. 2 Yeast plasmid pKFN1847 (Thim et al., FEBS Letters, 1993, 99: 345-352). The plasmid contains an expression cassette comprising an EcoRI-XbaI fragment inserted into the plasmid between the transcription-promoter (located on a SalI-EcoRI fragment) and the transcription-terminator of the S. cerevisiae TPI1 gene. POT is the selective marker, the Schizosaccharomyces pombe triosephosphate isomerase gene. AMP-R is an ampicillin resistance selection marker. Only restriction sites relevant for the construction of the plasmid described in example 1 have been indicated.

FIG. 3 Nucleotide sequence and corresponding amino acid sequence of the 563 bp sequence EcoRI-XbaI encoding the leader—K99-TFF2 fusion protein, described in example 1. The amino acids corresponding to the leader are framed. The N- and C-terminal amino acids of the mature K99-TFF2 are labelled 1 and 106, respectively.

FIG. 4 Reverse-phase HPLC on Vydac 214TP54 C4 column of supernatant from yeast strain YEA314 expressing glycosylated human K99-TFF2 and non-glycosylated human K99-TFF2. Absorbance was measured at 214 nm.

FIG. 5 Mass spectrometry analysis on a Voyager RP MALDI-TOF spectrometer of a sample of the non-glycosylated human K99-TFF2.

FIG. 6 Mass spectrometry analysis on a Voyager RP MALDI-TOF spectrometer of a sample of the glycosylated human K99-TFF2.

FIG. 7 Structures of three types of asparagine (Asn)-linked sugar chains, the high mannose-type, the complex-type and the hybrid-type. GlcNAc, NeuAc, Man, Fuc and Gal represents N-acetylglucosamine residues, N-acetylneuraminic acid residues, mannose sugar residues, fucose sugar residues and galactose sugar residues respectively. p₁, p₂, p₃ are integers independently selected from from 0 to 35, where p₁+p₂+p₃=35.

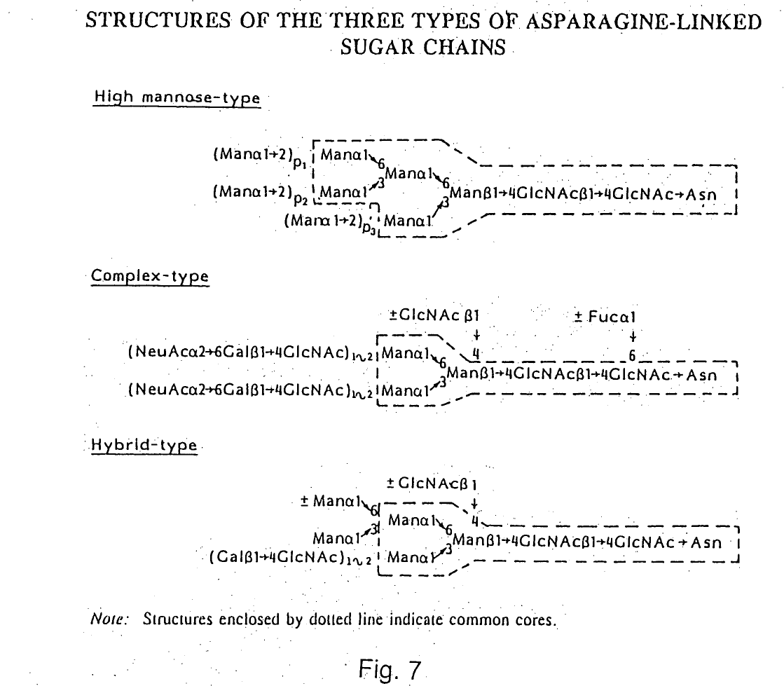

FIG. 8. Stress versus shear rate of mucin solution alone. 2 ml of 10% (w/v) mucin I dissolved in 0.05% (w/v) sodiumazide was added 0.4 ml of water. After 30 min at 20° C. the shear stress was measured as function of shear rate using the software programme: “constant rate—Approximation to power law.

The present invention is further illustrated by the following examples which, however, are not to be construed as limiting the scope of protection. The features disclosed in the fore-going description and in the following examples may, both separately and in any combination thereof, be material for realizing the invention in diverse forms thereof.

### EXAMPLES

### Example 1

Construction of a Yeast Expression System for K99-TFF2.

A Saccharomyces cerevisae expression system expressing a mutant hSP with an Asn at position 99 of the mature protein (hSP-Asn₉₉) has been described previously (Thim, L., 1993, FEBS Letters 318: 345-352).

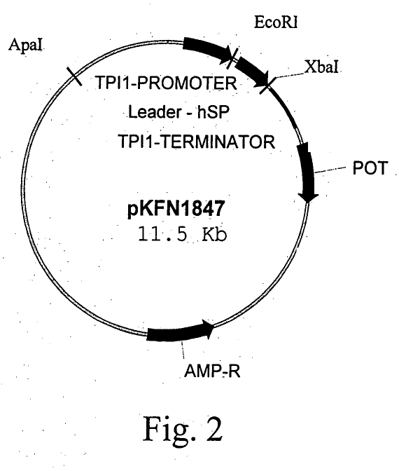

FIG. 2 shows a yeast plasmid called pKFN-1847 (Thim et al., FEBS Letters, 1993, 318: 345-352). The plasmid contains an expression cassette comprising an EcoRI-XbaI DNA fragment inserted into the plasmid between the transcription-promoter (located on a SalI-EcoRI fragment) and the transcription-terminator of the Saccharomyces cerevisiae TPI1 gene.

In plasmid pKFN-1847 the EcoRI-XbaI fragment encodes a fusion protein composed of a leader sequence, a Lys-Arg cleavage site for the dibasic processing endopeptidase KEX2, and the mutant hSP-Asn₉₉. In order to construct a plasmid encoding K99-TFF2, the following steps were performed using standard molecular biology techniques (e.g. Sambrook, J., Fritsch, E. F. and Maniatis, T., Molecular Cloning: A laboratory Manual, Cold Spring Harbour Laboratory Press, New York, 1989).

A 688 bp DNA fragment containing the EcoRI-XbaI DNA fragment and encoding the leader-hSP fusion protein was amplified with PCR from plasmid pKFN-1847 using oligonucleotides EA-ECO: (5′-CTA TTT TCC CTT CTT ACG-3′, SEQ ID NO:3) and E147: (5′-TM TCT TAG TTT CTA GAC TTA GTA ATG GCA GTC TCT CAC AGA CTT CGG GAA GAA GC-3′, SEQ ID NO:4). EA-ECO corresponds to a sequence located 114 bp upstream from the EcoRI site of the EcoRI-XbaI DNA fragment containing the expression cassette. E147 has been designed to introduce a single nucleotide mutation in the DNA sequence encoding hSP-Asn₉₉ changing Asn₉₉ of hSP-Asn₉₉ to Lys₉₉. After digestion with EcoRI and XbaI the DNA sequence encoding hSP-Lys₉₉, hereafter referred to as K99-TFF2, can thus be cloned as a EcoRI-XbaI DNA fragment.

The EcoRI-XbaI PCR fragment containing the DNA sequence encoding the leader—K99-TFF2 fusion protein was ligated to the ApaI-EcoRI DNA fragment of pMT742 (Egel-Mitani et al., Gene, 1988, 73: 113-120) containing the TPI1 promoter from S. cerevisiae and the ApaI-XbaI vector fragment of pMT742, resulting in plasmid pEA314. The plasmid pMT742 has a similar organization as pKFN-1847, and restiction sites are located as shown in FIG. 2.

The expression plasmid was propagated in E. coli, grown in the presence of ampicillin and isolated using standard techniques (Sambrook et al., 1989). The plasmid DNA was checked for insert by appropriate restriction nucleases (e.g. EcoRI, NcoI, ApaI, XbaI) and was shown by sequence analysis to contain the proper DNA sequence encoding K99-TFF2.

The plasmid pEA314 was transformed into S. cerevisiae strain MT663. Yeast transformants harbouring plasmid pEA314 were selected by glucose utilization as carbon source on YPD (1% yeast extract, 2% peptone, 2% glucose) agar (2%) plates. One transformant yEA314, was selected for fermentation.

Yeast strain yEA314 was cultivated at 30° C. for 72 hours in YPD media (Guthrie, C. & Fink, G. R., Eds., Guide to Yeast Genetics and Molecular Biology, Academic Press, 1991) with a final OD₆₀₀ of approximately 15-20. After centrifugation the cell pellet was discarded and the supernatant was used for further characterization of K99-TFF2.

S. cerevisiae strain MT663 (MATalMATαpep4-3/pep4-3 HIS4/his4tpi::LEU2/tpi::LEU2 Cir⁺) was used as host strain for transformation. Strain MT663 was deposited in the Deutsche Sammlung von Mikroorganismen und Zellkulturen in connection with filing WO 92/11378 and was given the deposit number DSM 6278. Transformation of MT633 was conducted as described in WO 98/01535

### Example 2

Purification of K99-TFF2

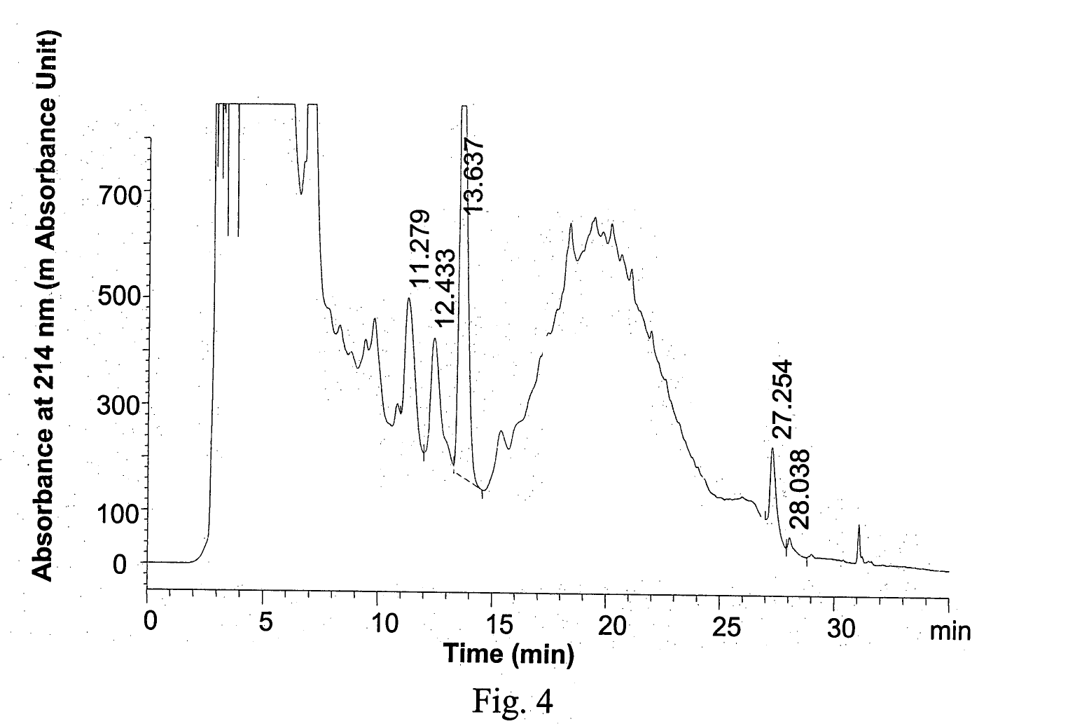

Yeast fermentation supernatant from yEA314 was concentrated from 2.5 ml to 0.25 ml using a Centricon® YM-3 3000 centrifugal filter device with the method described by the manufacturer (Millipore Corporation). The concentrated sample (0.25 ml) was injected onto a Vydac 214TP54 reverse-phase C4 HPLC column (0.46×25 cm) equilibrated at 25° C. at a flow rate of 1.0 ml/min with 0.1% (v/v) Trifluoroacetic acid in 10% (v/v) acetonitrile. After isocratic elution in 10 min the concentration of acetonitrile in the eluting solvent was raised to 60% (v/v) over 25 minutes. Absorbance was measured at 214 nm. The peaks eluting at 27.254 min. and 28.038 min. (FIG. 4) was found by mass spectrometry analysis to represent glycosylated human K99-TFF2 and non-glycosylated human K99-TFF2, respectively.

### Example 3

Characterization of K99-TFF2 by Mass Spectrometry

Mass spectrometric analysis was performed on a Voyager RP MALDI-TOF instrument (Perseptive Biosystems Inc., Framingham, Mass.) equipped with a nitrogen laser (337 nm). The instrument was operated in linear mode with delayed extraction, and the accelerating voltage in the ion source was 25 kV.

Sample preparation was done as follows: 1 μl sample-solution was mixed with 1 μl matrix-solution (alpha-cyano-4-hydroxy-cinnamic acid dissolved in a 5:4:1 (v/v/v) mixture of acetonitrile:water:3% (v/v) trifluoroacetic acid) and 1 μl was deposited on the sample plate and allowed to dry. Calibration was performed using two internal standards (insulin and thioredoxin) and the accuracy of the mass determinations was within 0.1%.

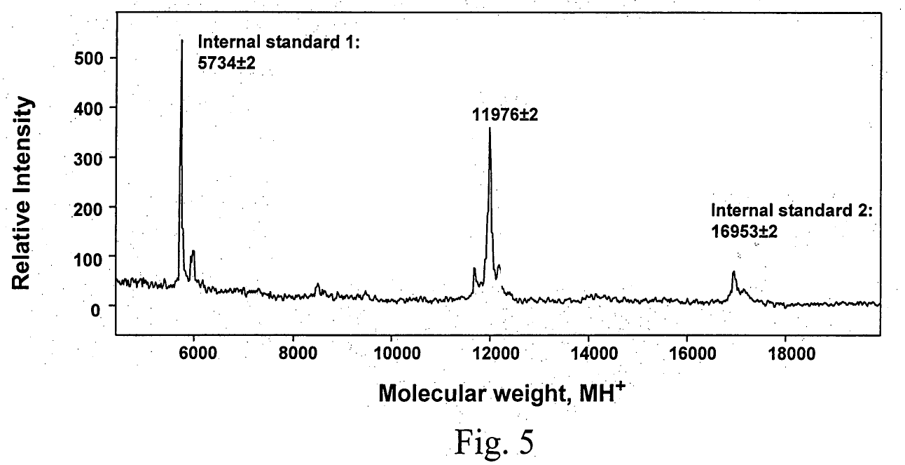

The results from the mass spectrometry analysis of the non-glycosylated human K99-TFF2 is shown in FIG. 5. An MH⁺-value of 11976±2 was found corresponding to a molecular weight of 11975±2. The molecular weight of human K99-TFF2 as calculated from the amino acid sequence is 11975.5. The experimental found molecular weight is thus in good agreement with the calculated value.

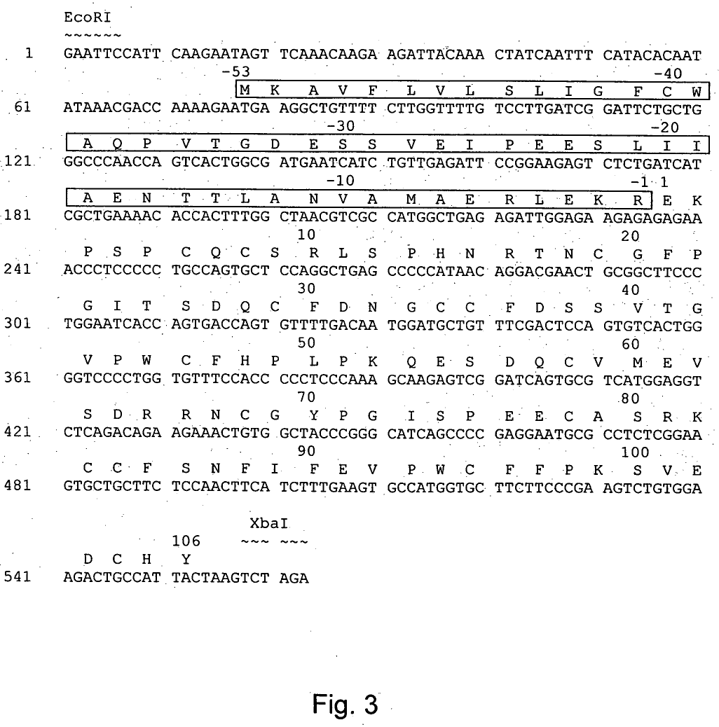

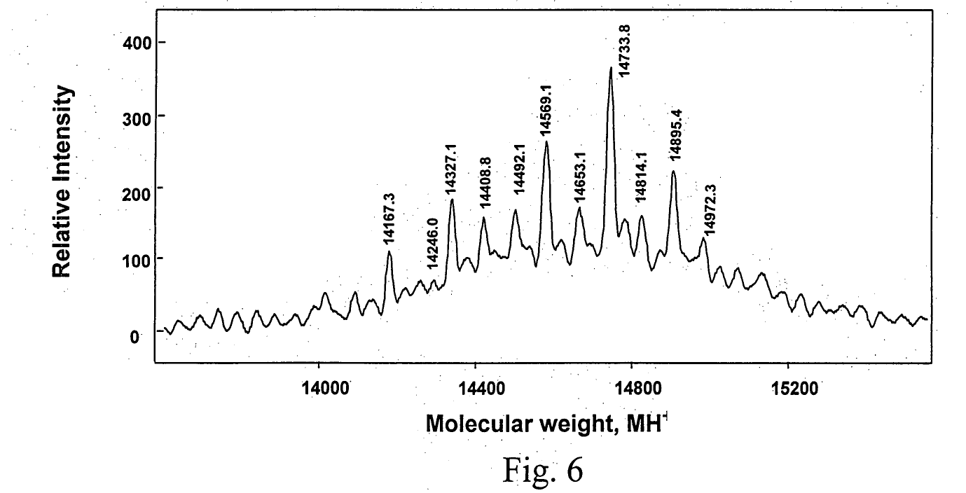

FIG. 6 shows the mass spectrometry analysis of the glycosylated human K99-TFF2.

| | Calculated | MW found by MS |
|---|---|---|
| Structure | MW | (See FIG. 3) |
| K99TFF2 + 2GlcNAc + 10Man | 14003.1 | (14005.0)* |
| K99TFF2 + 2GlcNAc + 11Man | 14165.2 | 14166.3 |
| K99TFF2 + 2GlcNAc + 12Man | 14327.4 | 14326.1 |
| K99TFF2 + 2GlcNAc + 13Man | 14489.5 | 14491.1 |
| K99TFF2 + 2GlcNAc + 14Man | 14653.7 | 14652.1 |
| K99TFF2 + 2GlcNAc + 15Man | 14813.8 | 14813.1 |
| K99TFF2 + 2GlcNAc + 16Man | 14971.3 | 14971.3 |
| K99TFF2 + 14Man | 14245.3 | (14245.0)* |
| K99TFF2 + 15Man | 14407.4 | 14407.8 |
| K99TFF2 + 16Man | 14569.5 | 14568.1 |
| K99TFF2 + 17Man | 14731.7 | 14732.8 |
| K99TFF2 + 18Man | 14893.9 | 14984.4 |

*Trace

From the amino acid sequence of the human K99-TFF2 it is known that there exists only one potential N-glycosylation site in the molecule (Asn-15). From previous studies of glycosylated TFF2 expressed in yeast (Thim et al. (1993) FEBS Left. 318, 345-352) it is known that only two monosaccharide residues, mannose and N-acetyl-glucosamine, occur in the glycosylated peptide. From the mass spectrometry data it is thus possible to deduce the different glycosylated forms of K99-TFF2 (above table). Molecular weights corresponding to two series of carbohydrate side chains can be deduced, namely (GlcNAc)₂(Hex)₁₀₋₁₅, and (Hex)₁₃₋₁₇. As mannose is the only hexose in the glycosylated peptide and Asn-15 is the only glycosylated residue it is concluded that the structure of the glycosylation site is either Asn-(GlcNAc)₂(Man)₁₀₋₁₅ or Asn-(Man)₁₃₋₁₇. It is possible that the Asn-(Man)₁₃₋₁₇ arise from fragmentation in the mass spectrometer, by which two GlcNAc residues lose an acetyl group and thereby are converted into two hexose residues.

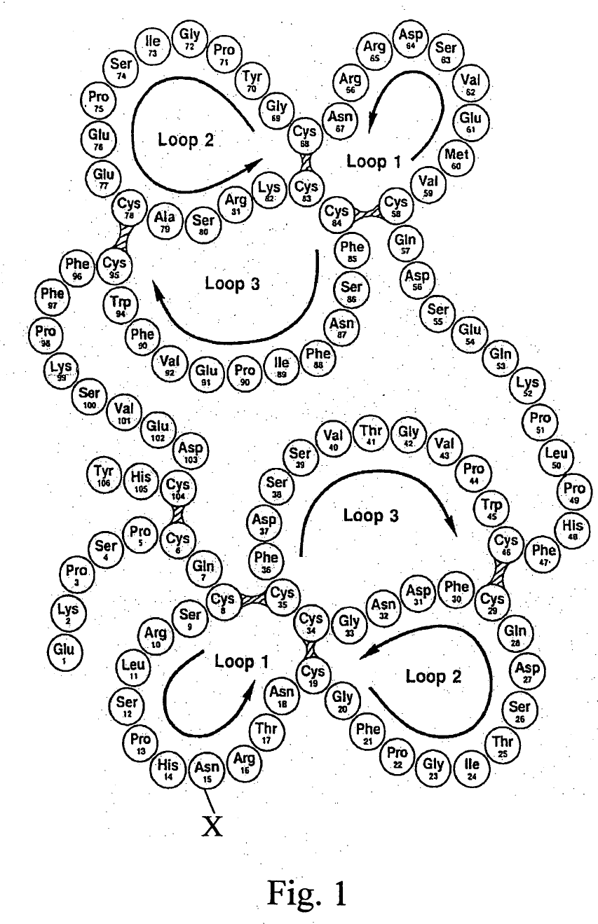

The structure of the glycosylated human K99-TFF2 is shown in FIG. 1.

### Example 4

Measurement of Rheological Properties of K99-TFF2/Mucin Complexes

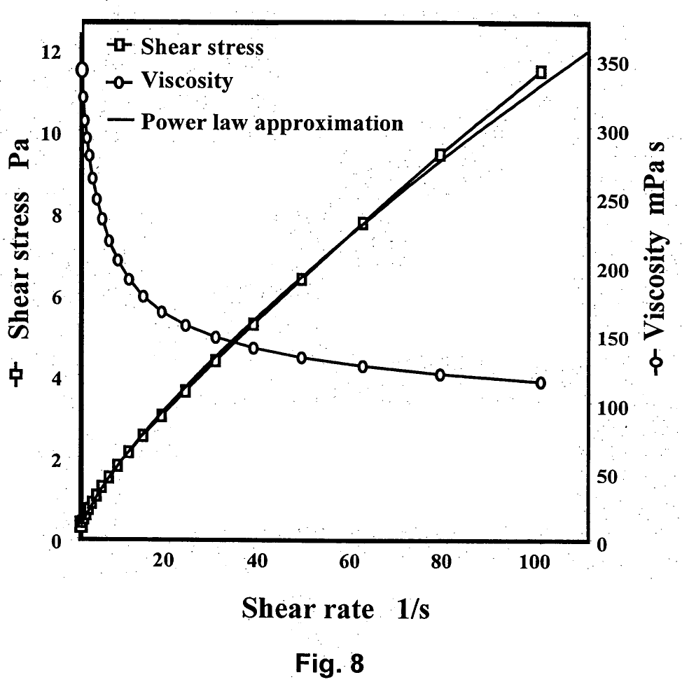

Mucin solutions and mucin/K99-TFF2 gel-like substances. Mucin solutions to which a K99-TFF2 peptide is added is compared. As can be seen from FIG. 8 the mucin solution alone behaves as a non-Newtonian liquid. These liquids can be described by the Ostwald de Waele model (power law) (Barnes, H. A. (1989) An introduction to rheology. Elsevier and Ferguson, J. and Kemblowski, Z. (1991) Applied fluid rheology. Elsevier):

δ=k(γ)ⁿ, where δ=shear stress, γ=shear rate and n and k are constants specific for the solution (if n=1 the solution is Newtonian). In the present case the following values could be calculated from FIG. 8: n=0.75 and k=0.35.

Since n<1 the solution is called shear-tinning, which is the characteristics of dispensions with asymmetric particles or emulsions. However, since the n value is close to 1 the solution is not far from being Newtonian. As can also be seen from FIG. 8 the viscosity varies from 0.34 Pa s at low shear rates to 0.12 Pa s at high shear rates.

K99-TFF2 has the properties of forming highly viscous complexes with mucins. The rheological properties of such complexes are measured by the use of a rotational Reologica Rheometer (Reologica Instruments AB, Lund, Sweden). The instrument is equipped with a stainless steel C40 4 cone-plate (40 mm diameter plate with an angle of 4 degree and zero gab) requiring a sample volume of 1.17 ml. All rheological experiments are carried out at temperature of 20° C. The rheometer is operated using instrument standard software (Version 3.6) allowing several different types of measurements. Two basic types of rheological measurements are performed. Viscosity determination is carried out using a Constant Rate program in which the stress and hence the viscosity is determined as a function of shear rate. The shear rate range is set to 0-20 s⁻¹.

In further evaluating the rheological experiments dynamic oscillatory is employed. For this purpose an Oscillation Stress Sweep program is used to identify the linear viscoelastic region (LVER), i.e. the stress range in which the measurement results are independent of the applied stress. An appropriate stress value representative of LVER is then chosen for the Oscillation program where the rheological behavior can be determined at different frequencies (frequency sweeps). The frequency sweeps are conducted in the frequency range from 0.01-5 Hz.

Three types of mucins are used. Mucin l: Crude mucin, type II from porcine stomach (Sigma, St. Louis, Mo., USA). Mucin II: Partially purified mucin, type III from porcine stomach (Sigma, St. Louis, Mo., USA). Mucin III: mucin, type I-S from bovine submaxillary glands (Sigma, St. Louis, Mo., USA).

In experiments for visual assessment of the change in viscosity a 10% (w/v) solution of mucin I was prepared and K99-TFF2 peptides were dissolved in water and added to the mucin solution. After mixing the sample (Vortex mixer), the sample was allowed to stand for 5 min. and the viscosity was visually assessed in relation to a control solution of mucin added water without K99-TFF2. A visual assessment of the change in properties that can be observed when different K99-TFF2 peptides is added to mucin solutions is made (Table 1). In some experiments the effect was astonishing. The addition of K99-TFF2 peptides to mucin solutions resulted in thick gel-like substance that did hardly leave the test-tube even if the tube was turned bottom up.

All mucin/trefoil mixtures for rheological examination are prepared using the Vortex mixer and allowed to equilibrate at 20° C. for 30 minutes after which the viscosity is measured.

As several mucin preparations with different characteristics are commercially available it is first established which mucin type would be suitable and in which concentration. A fixed amount of K99-TFF2 peptide (7 mg) is added to 2 ml Y % (w/v) mucin I solution. The Y is varied from 6%, 8%, 10%, 12% and 14% (w/v). No mucin/K99-TFF2 gel-like structure is normally formed with the 6% and 8% mucin solution, but a fibre-like precipitate surrounded by liquid mucin solution is formed. Using the 10%, 12% and 14% mucin solution the mucin/K99-TFF2 gel-like structure is formed. A 10% mucin concentration can then be chosen for further experiments.

To further evaluate the rheological properties of the mucin/K99-TFF2 gel-like structure the technique of oscillatory rheology is employed. Dynamic oscillatory rheology is generally considered a non-destructive method measuring delicate viscoelastic aspects of a material.

In order to characterise the mucin/K99-TFF2 gel-like structure the material is subjected to a sinusoidally varying stress and the strain response was measured. Initially an oscillation stress sweep programme to define the so-called linear viscoelastic region is used. Inside this region no change of the mucin/K99-TFF2 structure occurs and the relation between the applied stress and the measured quantities is linear.

The viscoelasticity is described by the dynamic moduli, G′ and G″ as a function of frequency, where G′ is the elastic (storage) modulus and G″ the viscous (loss) modulus. The storage modulus (a measure of the energy stored and recovered per cycle of deformation) reflects the solid-like component of viscoelastic behaviour of the material, while the loss modulus (a measure of the energy lost per cycle) reflects the liquid-like component. Furthermore, the complex viscosity and tan delta are determined. The complex viscosity is a measure of the magnitude of the total resistance to a dynamic shear. Tan delta is G″/G″, where tan delta>1 reflects a more viscous material, and tan delta<1 indicates a more elastic material.

Oscillatory measurement of mucin solution and mucin/K99-TFF2 gel-like material is carried using the following procedure: 2 ml of 10% (w/v) mucin I dissolved in 0.05% (w/v) sodiumazide is added 0.4 ml of water 0.4 ml of water containing 14 mg K99-TFF2. After 30 min at 20° C. a sinosoidally varying stress was applied and the strain response is detected at different frequencies. The elastic modulus (G′) (with and without TFF) and the viscous modulus (G″) (with and without TFF) is calculated as a function of different frequencies.

TABLE 1
Visual assessment of viscosity

| | K99-TFF2 | Amount | Viscosity |
|---|---|---|---|
| Mucin I solution | peptide | K99-TFF2 added | increase |
| 1 ml 10%(w/w) | Lys99-TFF2 | 10 mg in 200 μl | +++++ |
| 1 ml 10%(w/w) | None | 0 | 0 |

### Example 5

Measurement of Plasma Half-Life.

A dose between 0.5 and 5 nmol/kg in 2 ml of K99-TFF2 is given to a pig by s.c. injection. Blood samples are drawn from an ear vein at the following times: pre-dose, 0, 15, 30, 45, 90, 120, 180, 240, 300, 360, 480 and 1440 minutes post injection.

Blood samples are collected into tubes containing 35 μl stabilization buffer per ml blood. The stabilization buffer consisted of: EDTA (di-sodium) 0.18 M and Aprotinin 15000 KIE/ml. The solution is pH adjusted to 7.4. Blood samples are kept on ice for no longer than 20 min. before centrifugation (4° C., 4000 rpm, 10 min). After centrifugation plasma is isolated and frozen at −20° C. until assayed. Plasma samples are analyzed by RIA or ELISA. Plasma concentration-time profiles are obtained from K99-TFF2 peptides for comparison. Data is analyzed by a non-compartmental pharmacokinetic analysis in Win Nonlin Professional, version 3.1, Pharsight Corporation. The following pharmacokinetic parameters are estimated:
Cₘₐₓ: Maximum observed plasma concentration
Tₘₐₓ: Time of maximum observed plasma concentration
t_{1/2}: Terminal plasma elimination half-life
AUC₍₀₋ₗₐₛₜ₎: Area under the serum concentration-time curve from time 0 to the time of the last measurable observation.
AUC: Area under the plasma concentration-time curve from time 0 extrapolated to infinity.

### Example 6

Measurement of Stability in the Gastrointestinal Tract

A dose between 0.5 and 50 nmol/kg K99-TFF2 is given in 50 ml water to a pig by intra gastric installation. Gastric juice samples are drawn at the following times: pre-dose, 0, 15, 30, 45, 90, 120, 180, 240, 300, 360, 480 and 1440 minutes post installation. Gastric samples are analysed by RIA or ELISA. Gastric concentration-time profiles from K99-TFF2 peptides is obtained. Data is analyzed as described in example 5.

What we claim is:

1. A composition comprising one or more trefoil factor 2 (TFF2) peptides, wherein each of said peptides (i) has an amino acid sequence corresponding to SEQ ID NO:1, and (ii) has a moiety X covalently attached to Asn15, wherein said X is independently selected from sugar residues and oligosaccharides.

2. The composition according to claim 1, wherein the trefoil factor 2 (TFF2) peptide with an amino acid sequence of SEQ ID NO:1 further comprises disulphide bonds between Cys6-Cys104, Cys8-Cys35, Cys19-Cys34, Cys29-Cys46, Cys58-Cys84, Cys68-Cys83, and Cys78-Cys95.

3. The composition according to claim 1, wherein X is a sugar residue.

4. The composition according to claim 1, wherein X is independently selected from (Hex)ₙ or (GlcNAc)₂-Y, wherein n is an integer from 1 to 40 and wherein Y is a sugar residue.

5. The composition according to claim 1, wherein X is (Hex)ₙ and wherein n is an integer from 1 to 40.

6. The composition according to claim 5, wherein n is an integer from 13-17.

7. The composition according to claim 1, wherein X is (GlcNAc)₂-Y.

8. The composition according to claim 1, further comprising at least one peptide wherein X is (Hex)ₙ and at least one peptide wherein X is (GlcNAc)₂-Y, wherein n is an integer for 1 to 40 and Y is a sugar residue.

9. The composition according to claim 7, wherein Y is (Hex)ₙ, and n is an integer from 1 to 40.

10. The composition according to claim 9, wherein n is an integer from 10 to 15.

11. The composition according to claim 7, wherein Y is (Hex)ₙ( (GlcNAc)(Hex) )ₘ, and n and m are integers independently selected from 1 to 40.

12. The composition according to claim 7, wherein Y is (Hex)ₙ( (GlcNAc)(Gal) )ₘ, and n and m are integers independently selected from 1 to 40.

13. The composition according to claim 7, wherein Y is (Hex)ₙ( (GlcNAc)(Hex)(NeuAc) )ₘ, and n and m are integers independently selected from 1 to 40.

14. The composition according to claim 7, wherein Y is (Hex)ₙ( (GlcNAc)(Gal)(NeuAc) )ₘ, and n and m are integers independently selected from 1 to 40.

15. The composition according to claim 4, wherein Hex is mannose.

16. A composition comprising one or more trefoil factor 2 (TFF2) peptides, wherein each of said peptides (i) has an amino acid sequence corresponding to SEQ ID NO:1, and (ii) has a moiety X covalently attached to Asn15, wherein said X is characterized by the glycosylations produced by expression of the trefoil factor 2 TFF2 peptides in a eucaryotic host cell.

17. The composition according to claim 16, wherein the host cell is yeast.

18. The composition according to claim 17, wherein the yeast is Saccharomyces cerevisiae.

19. The composition according to claim 16, wherein the host cell is a mammalian cell line.

20. The composition according to claim 19, where the mammalian cell line is a human cell line.

21. The composition according to claim 16, wherein the host cell is an insect cell line.

22. A pharmaceutical composition comprising one or more trefoil factor 2 (TFF2) peptides, wherein each of said peptides (i) has an amino acid sequence corresponding to SEQ ID NO:1, and (ii) has a moiety X covalently attached to Asn15, wherein said X is independently selected from sugar residues and oligosaccharides, and a pharmaceutically acceptable carrier or diluent.

23. A pharmaceutical composition for increasing the viscosity of mucus layers or for the treatment of damaged or abnormal mucus layers in mammals, comprising the composition according to claim 1 or a pharmaceutically acceptable salt thereof, together with a pharmaceutically acceptable carrier or diluent.

24. The pharmaceutical composition according to claim 22, wherein the pharmaceutically acceptable carrier or diluent is suitable for oral administration, buccal administration, nasal administration, ocular administration, transdermal administration, pulmonary administration, parenteral administration, local application or luminal application.

25. The pharmaceutical composition according to claim 22, wherein the composition further comprises a mucin glycoprotein preparation.

26. The pharmaceutical composition according to claim 22, wherein the pharmaceutical composition is in eye droplets.

27. A method for preparing the composition according to claim 1, the method comprising culturing a eucaryotic host cell transformed with a DNA sequence encoding a trefoil factor 2 (TFF2) peptide under conditions permitting glycosylation, and recovering the resulting trefoil factor 2 (TFF2) peptides from the culture.

28. A method for preparing the composition according to claim 2, the method comprising culturing a eucaryotic host cell transformed with a DNA sequence encoding a trefoil factor 2 (TFF2) peptide under conditions permitting glycosylation, and recovering the resulting trefoil factor 2 (TFF2) peptides from the culture.

29. A DNA construct containing a nucleotide sequence encoding human trefoil factor 2 (TFF2) peptide having the amino acid sequence of SEQ ID NO:1.

30. The DNA construct according to claim 29, wherein said nucleotide sequence also encodes a leader peptide and a Lys-Arg cleavage site.

31. The DNA construct according to claim 29, wherein said nucleotide sequence comprises the cDNA sequence of SEQ ID NO:2.

32. A recombinant vector capable of transforming in a host cell, wherein said vector contains a nucleotide sequence encoding human trefoil factor 2 (TFF2) peptide having the amino acid sequence of SEQ ID NO:1, a promoter for host cell propagation and a selection marker.

33. The recombinant vector according to claim 32, wherein the host cell is yeast.

34. The recombinant vector according to claim 33, wherein the yeast is Saccharomyces cerevisiae.

35. The recombinant vector according to claim 32, wherein the host cell is a bacteria.

36. The recombinant vector according to claim 32, wherein the host cell is an insect cell.

37. The recombinant vector according to claim 32, wherein the host cell is a mammalian cell.

38. The recombinant vector according to claim 37, wherein the mammalian cell a human cell.

39. The recombinant vector according to claim 32, wherein the recombinant vector is a DNA plasmid.

40. A yeast cell transfected with the recombinant vector according to claim 32.

41. A method for increasing the viscosity of mucus layers in a subject in need thereof, said method comprising administering to the subject a composition comprising:
(a) one or more trefoil factor 2 (TFF2) peptides, wherein each of said peptides (i) has an amino acid sequence corresponding to SEQ ID NO:1, and (ii) has a moiety X covalently attached to Asn15, wherein said X is independently selected from sugar residues and oligosaccharides;
(b) a pharmaceutically acceptable carrier or diluent; and
(c) optionally, a mucin glycoprotein preparation.

42. The method according to claim 41, wherein the administration is local, luminal, or parenteral.

43. The method according to claim 41, wherein the mucus viscosity levels are associated with a disease state in the oral mucosa, the respiratory passages, the distal part of the oesophagus, the stomach, the small intestine or colon, an eye, a joint, or the urinary system.

44. The method according to claim 43, wherein the disease state in the oral mucosa is a reduced secretion of saliva.

45. The method according to claim 44, wherein the reduced secretion of saliva is caused by irradiation therapy, treatment with anticholinergics or Sjögrens syndrome.

46. The method according to claim 41, wherein the disease state in the respiratory passages is selected from rhinorrhoea, the common cold, allergic rhinitis, or the accidental inhalation of irritants, gases, dusts or fumes.

47. The method according to claim 41, wherein the disease state is in the stomach is selected from acid reflux oesophagitis, hiatus hernia, Barrets oesophagus, stress induced gastric ulcers, or diarrhoea.

48. The method according to claim 41, wherein the disease state in the small intestines if colon is selected from Crohn's disease or ulcerative colitis.

49. The method according to claim 41, wherein the disease state in the eye is keratoconjunctivitis sicca/Sjögren's syndrome or dry eyes.

50. The method according to claim 41, wherein the disease state in a joint is an increase in the viscosity of the synovial fluid.

51. The method according to claim 41, wherein the disease state in the urinary tract is selected from chronic bladder infection, catheterisation, interstitial cystitis, papillomas or cancer of the bladder.
